(19) United States
(12) Patent Application Publication
(10) Pub. No.: US 2022/0143086 A1
(43) Pub. Date: May 12, 2022

(54) METHOD OF HOMING AND RETENTION OF GAMMADELTA T CELLS, OPTIONALLY WITH NATURAL KILLER CELLS, FOR GENERATING CELL COMPOSITIONS FOR USE IN THERAPY

(71) Applicant: Gamida Cell Ltd., Jerusalem (IL)

(72) Inventor: Tony PELED, Mevaseret Zion (IL)

(21) Appl. No.: 17/432,758

(22) PCT Filed: Feb. 24, 2020
(86) PCT No.: PCT/IL20/50206

§ 371 Date: Aug. 20, 2021

Related U.S. Application Data
(60) Provisional application No. 62/809,671, filed on Feb. 24, 2019.

(51) Int. Cl. A61K 35/17; A61K 35/28; C12N 5/0783

(52) U.S. Cl. CPC A61K 35/17; A61K 35/28; C12N 5/0636; C12N 2501/2321; C12N 2500/30; C12N 2501/2302; C12N 2501/2315; C12N 5/0646

(57) ABSTRACT

Methods of ex-vivo culture of gammadelta T-cells and gammadelta T-cell enriched cell populations are provided and, more particularly, methods for enhancing functionality of gammadelta T-cell populations by treating the cells with a nicotinamide in combination with cytokines enhancing gammadelta T-cell homing and/or retention potential. Also envisioned are compositions comprising cultured gammadelta T-cells and mixed gammadelta T-cell enriched cell populations and therapeutic uses thereof.

### RELATED APPLICATION

This application claims the benefit of priority of U.S. Provisional Patent Application No. 62/809,671 filed on 24 Feb. 2019, the contents of which are incorporated herein by reference in their entirety.

### FIELD AND BACKGROUND OF THE INVENTION

The present invention, in some embodiments thereof, relates to methods for culturing gammadelta T-cell populations, therapeutic use of cultured gammadelta T-cell populations, and kits comprising the cultured gammadelta T-cells. More particularly, but not exclusively, the present invention relates to the use of cultured gammadelta T-cells, alone or in combination with other cells for transplantation.

Gammadelta (γδ) T-cells are a conserved population of innate lymphocytes taking part in numerous immune responses during tissue homeostasis, infectious disease, autoimmune disease, inflammation, transplantation and tumor surveillance. Gammadelta T-cells can share attributes of the adaptive or innate immune system, or of both, and comprise a thymus and peripheral tissue sub-set recognizing stress-related antigens (Vδ1 cells), a circulating sub-set activated by phosphoantigens (Vδ2 cells) and a sub-set found mostly in the liver (Vδ3 cells) and common in viral infections and leukemia.

When activated, gamma delta T cells exert potent, non-MHC restricted cytotoxic activity, especially efficient at killing various types of cells, particularly pathogenic cells, such as infected (viral, parasitic, fungal, etc. infections) and cancer cells. Gammadelta T-cells have been implicated in the long-term survival of hematopoietic stem cell transplantation patients, and their presence in tumor samples has been identified as a significant favorable cancer prognostic signature. Gammadelta T-cells are able to sense altered lipid pathways, detecting and eliminating malignant cells irrespective of the tumor antigen signature, and are highly chemoresistant, making them particularly suited for combination immunochemotherapy.

Gammadelta T-cells constitute only a small percentage of human peripheral blood and tissue-residing T cells (1-5%), and even a lower percentage (<1%) of umbilical cord lymphocytes. Thus, therapeutic application of gammadelta T-cell populations requires means for their expansion. The two main approaches to the enrichment of gammadelta T-cells for clinical use (e.g. in cancer immunotherapy) include in-vivo expansion of endogenous gammadelta populations by administering stimulating phosphoantigens or amino bisphosphonates together with low dose recombinant IL-2, and ex-vivo adoptive cell transfer of in vitro expanded gamma delta T cells (autologous or heterologous) into a patient, (see, for example, US Patent Application 2017/0196910 to Leeks et al). Due to the undesirable side effects and brief serum half-life of IL-2, and disappointing results in clinical trials of in-vivo IL-2 administration, ex-vivo expansion of gammadelta T-cells is the currently preferred method. Some studies have also indicated that IL-15 can be effective in promoting proliferation, survival and cytotoxicity of gammadelta T-cells.

Enhancing functionality of gammadelta T-cells is critical to effective T-cell therapy. Gammadelta T-cells are activated by, inter alia, small compounds such as the non-specific HMB-PP; and aminobisphosphonates, accumulating in cancer, and cellular stress proteins such as annexin A2. Some cytokines of the gamma-chain family, in addition to IL-2 (in particular, IL-7, IL-15 and IL-21) can also increase proliferation, survival and cytotoxicity of gammadelta T-cells (Van Acker et al, Cytokine and Growth Factor Rev 2018 41:54-64). Gammadelta T-cells also possess integrins (e.g. beta1, beta2, beta7 integrin, vitronectin receptor) which function in the homing, adhesion, signaling, migration, infiltration and retention of gammadelta T-cells in tissues following transplantation (Seigers, Front in Immunol, 2018). Gammadelta T-cells also express L-selectin (CD62L) and E- and P-selectin ligands, as well as other homing molecules in response to inflammation, mediating their homing and retention in various tissue types (skin, gut, liver, brain, bone marrow, lymph nodes, etc.) (Sackstein et al, Lab Invest 2017 97:669-97).

Methods for ex-vivo culture of gammadelta T-cells for transplantation have been proposed. For example, US2005/0196385 to Romagne and Laplace and US2009/0130074 to Moser and Kuchen teach expansion and activation of gammadelta T-cells using synthetic or natural gammadelta T-cell activator small molecules. US2017/0107490 to Maeurer and US2018/0312808 to Hayday et al, teach the ex-vivo expansion of gammadelta T-cells by culturing with different combinations of IL-2, IL-15 and IL-21. Some models of gammadelta T-cell expansion include induction or engineering of antigen-presenting functions in the gammadelta T-cells (see, for example, US2008/0075732 to Moser and Kuchen), engineering of expression of tumor recognition moieties (see, for example, Jakobovits et al, US2016/0175358), selection of gammadelta T-cells from CAR (chimeric antigen receptor)-expressing pluripotent stem cells (for example: US2016/0009813 to Themeli et al, US2018/0353588 to Boyd et al, 2018/0125889 to Leek et al, US2018/0200299 to Cooper et al, US2018/0250337 to Lamb et al), directing HSC to differentiate into gammadelta T-cells (for example, US2016/0213715 to Messina and Tie) and engineering of CXCR6 expression in the gammadelta T-cells (US2018/0256645 to Kobold et al) for targeting to tumors.

Therapeutic use of expanded populations of gammadelta T-cells has been the subject of more than 15 completed, active, recruiting or authorized clinical trials (see clinical trials (dot)gov website) investigating application of gammadelta T-cells expanded by different protocols for the treatment of a variety of infectious and cancerous conditions. Expanded gammadelta T-cell populations have been found, in general, to maintain cytotoxic function. However, results to date underscore the difficulty in designing gammadelta T-cell expansion and therapy protocols that are not only safe but provide large, expanded populations of gammadelta T-cells sufficiently effective in targeting the affected tissues.

Additional relevant publications include Nicol et al (BJ of Cancer, 2011 105:778-106), Kobayashi et al (AntiCancer Res 2010 30:575-580), Berglund et al (Stem Cell Int 2018 ID8529104), Tan et al (J Immunol Sci 2018 2:6-12), Fisher et al (Frontiers in Immunol 2018 9:1409), US20018/0207568 to Belmant and US2009/0304688 to Fournie et al.

### SUMMARY OF THE INVENTION

According to one aspect of the present invention there is provided a method of enhancing gammadelta T-cell homing and/or retention potential, the method comprising:

(a) obtaining a selected cell population enriched for gammadelta T-cells;
(b) ex-vivo providing the selected cell population with conditions for gammadelta T-cell expansion,
(c) providing nicotinamide in the range of 0.5 to 50 mM for a period of time sufficient for enhancing gammadelta T-cell homing and/or retention potential, thereby enhancing homing and/or retention potential of gammadelta T-cells in the selected cell population.
According to one aspect of the present invention there is provided a method of enhancing gammadelta T-cell CD62L expression, the method comprising:
(a) obtaining a selected cell population enriched for gammadelta T-cells;
(b) ex-vivo providing the selected cell population with conditions for gammadelta T-cell expansion,
(c) providing nicotinamide in the range of 0.5 to 50 mM for a period of time sufficient for enhancing gammadelta T-cell CD62L expression, thereby enhancing CD62L expression of gammadelta T-cells in the selected cell population.

According to still further features in the described preferred embodiments the conditions for gammadelta T-cell expansion comprise providing nutrients and cytokines.

According to still further features in the described preferred embodiments the cytokines are selected from the group consisting of IL-2, IL-15 and IL-21.

According to still further features in the described preferred embodiments the nicotinamide is selected from the group consisting of nicotinamide, a nicotinamide analog, a nicotinamide metabolite, a nicotinamide analog metabolite and derivatives thereof.

According to still further features in the described preferred embodiments the selected cell population is a lymphocyte cell population enriched for gammadelta T-cells by alphabeta T-cell depletion.

According to still further features in the described preferred embodiments the selected cell population comprises natural killer (NK) cells.

According to still further features in the described preferred embodiments the method further comprises providing conditions for NK cell expansion.

According to still further features in the described preferred embodiments providing the conditions for gammadelta T-cell expansion and the nicotinamide enhances homing and/or retention potential and/or CD62L expression of the NK cells in the selected cell population.

According to still further features in the described preferred embodiments the selected cell population is a lymphocyte cell population enriched for gammadelta T-cells by selection of gammadelta T-cells.

According to still further features in the described preferred embodiments the selected cell population is devoid of NK cells.

According to still further features in the described preferred embodiments the population of gammadelta T-cells is derived from an organ selected from the group consisting of a muscle, skin, a bone, a lymph organ, a pancreas, a liver, a gallbladder, a kidney, a digestive tract organ, a respiratory tract organ, a reproductive organ, a urinary tract organ, a blood-associated organ, a thymus, a spleen, a nervous system organ.

According to still further features in the described preferred embodiments the population of gammadelta T-cells is derived from a source selected from the group consisting of hematopoietic cells, umbilical cord blood cells, mobilized peripheral blood cells and bone marrow cells.

According to still further features in the described preferred embodiments the population of gammadelta T-cells is derived from bone marrow or peripheral blood.

According to still further features in the described preferred embodiments the population of gammadelta T-cells is derived from neonatal umbilical cord blood.

According to still further features in the described preferred embodiments the population of cells is derived from a mononuclear cell fraction.

According to still further features in the described preferred embodiments the population of gammadelta T-cells is from an apheresis sample.

According to still further features in the described preferred embodiments the period of time of step (c) of the method is between 1 and 3 weeks.

According to still further features in the described preferred embodiments the period of time of step (c) of the method is between 1 and 7 days.

According to still further features in the described preferred embodiments the concentration of the nicotinamide is in the range of 0.5-20 mM.

According to still further features in the described preferred embodiments the nicotinamide is provided at a concentration of 5 mM.

According to still further features in the described preferred embodiments the method further comprising selecting a gammadelta T-cell population according to a cell marker selected from the group consisting of a tumor antigen, a viral antigen and a bacterial antigen.

According to one aspect of the present invention there is provided a therapeutic cell composition comprising an expanded selected gammadelta T-cell population, the cell population ex-vivo cultured with conditions for gammadelta T-cell expansion and amount of nicotinamide in the range of 0.5-50 mM, wherein the expanded selected gammadelta T-cell population is characterized by at least one of:

(i) enhanced gammadelta T-cell homing and/or retention potential, and

(ii) enhanced expression of CD62L,

as compared to a similar selected gammadelta T-cell population expanded with identical conditions and no more than 0.1 mM nicotinamide.

According to still further features in the described preferred embodiments the therapeutic cell composition comprises gammadelta T-cells cultured according to the method of the invention as detailed herein.

According to still further features in the described preferred embodiments the therapeutic cell composition further comprises NK cells.

According to one aspect of the present invention there is provided a method of transplanting cells in a subject, the method comprising:

(a) ex-vivo expanding a selected gammadelta T-cell population by culturing the cell population conditions for gammadelta T-cell expansion and nicotinamide in the range of 0.5-50 mM for a period of time sufficient for enhancing gammadelta T-cell homing and/or retention potential and/or CD62L expression, wherein the expanded selected gammadelta T-cell population is characterized by at least one of:

(i) enhanced gammadelta T-cell homing and/or retention potential, and

(ii) enhanced CD62L expression,

as compared to a similar selected gammadelta T-cell population expanded without 0.5-50 nM nicotinamide, and

(b) infusing the expanded gammadelta T-cells into a subject in need thereof.

According to still further features in the described preferred embodiments step (a) of the method of transplanting cells is affected according to the method of gammadelta T-cell expansion of the invention as described herein in detail.

According to still further features in the described preferred embodiments the subject is a human subject.

According to still further features in the described preferred embodiments the gammadelta T-cells are allogeneic to the subject.

According to still further features in the described preferred embodiments the gammadelta T-cells are autologous to the subject.

According to still further features in the described preferred embodiments the subject is suffering from a condition selected from the group consisting of a cancer, a bacterial infection, a viral infection, an autoimmune condition and an inflammatory condition.

According to still further features in the described preferred embodiments the transplantation of the cells in the subject comprises an adjunct therapy.

According to still further features in the described preferred embodiments the adjunct therapy is in combination with a therapy selected from the group consisting of anti-viral therapy, anti-inflammatory therapy, antibiotic therapy, bactericidal therapy, chemotherapy, surgery, immunotherapy, immunochemotherapy, radiotherapy, bone marrow transplantation and hematopoietic stem cell transplantation.

According to still further features in the described preferred embodiments the subject is being treated with umbilical cord blood hematopoietic stem cells expanded in culture with greater than 1.0 mM nicotinamide prior to, concomitantly with or following transplantation of the expanded gammadelta T-cells.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a histogram showing the enrichment of gammadelta T cells in alphabeta-depleted peripheral blood cell samples. Alphabeta-depleted blood cells were cultured with or without 5 mM nicotinamide (NAM) for 12-13 days, CD3+ cells selected, and analyzed by FACS for CD3+/gammadelta+ and CD3+/alphabeta+ cells. Note greater than 90% gammadelta T cells in these T-cell fractions of both NAM and control cultures;

FIG. 2 is a histogram showing enhancement of CD62L (L-selectin) expression in gammadelta T cells by nicotinamide. Purified gammadelta T cells from alphabeta-depleted blood cells cultured 12-13 days with 5 mM nicotinamide (NAM) were stained for CD62L and analyzed by FACS. Note the nearly 3 fold increase in CD62L expression in cultures treated with nicotinamide;

FIG. 3 is a histogram showing enhancement of functionality of gammadelta T cells by culture by nicotinamide. Purified gammadelta T cells from alphabeta-depleted blood cells expanded with or without 5 mM nicotinamide (NAM) and labeled with CFSE were injected into irradiated NSG immunodeficient mice. Fractions of the CFSE stained cells from various organs were evaluated by FACS after 4 days. Note the striking effect of NAM on in-vivo homing and tissue retention of the gammadelta cells in all tissues analyzed.

### DESCRIPTION OF SPECIFIC EMBODIMENTS OF THE INVENTION

The present invention, in some embodiments thereof, relates to methods for culturing gammadelta T-cell populations, therapeutic use of cultured gammadelta T-cell populations, and kits comprising the cultured gammadelta T-cells. More particularly, but not exclusively, the present invention relates to the use of cultured gammadelta T-cells, alone or in combination with other cells for transplantation.

Nicotinamide (NAM), the amide form of niacin (niacinamide, Vitamin B3) is a base-exchange substrate and a potent inhibitor of NAD(+)-dependent enzymes endowed with mono- and poly-ADP-ribosyltransferase activities. As a trace micronutrient, nicotinamide is required, in micro-molar amounts, for assuring viability and proliferation of mammalian cells in ex-vivo culture, and is commonly included, along with other vitamins in formulae for cell culture media. Like other lymphocyte fractions, gammadelta T-cells are typically ex-vivo cultured in medium comprising nicotinamide in concentrations ranging from about 8 μM nicotinamide (MEMα, RPMI) to about 33 μM (DMEM) to promote robust gammadelta T-cell growth (see, for example, US Patent Application 2016/0175358 to Jakobovits et al).

Higher concentrations of nicotinamide have been found effective for enhancing expansion and functionality of CD34+ and CD133+ hematopoietic stem and progenitor cells (see, for example, U.S. Pat. Nos. 7,955,852 and 8,846,393) and CD56+ natural killer cells (see, for example, Frei et al, Blood 2011 118:4035), but have also been reported to inhibit responsiveness to activation signals and induce apoptosis in T-cells (see, for example, Liu et al J Immunol 2001 167:4942-4947) and neutrophils (Fernandes et al, Am J Physiol Lung Cell Mol Phys 2011 300: L354-361).

The present inventors have surprisingly shown that addition of millimolar concentrations of nicotinamide to enriched populations of human gammadelta T-cells ex-vivo cultured in the presence of conditions for gammadelta T-cell proliferation, as is further detailed herein, results in enhanced functionality, e.g. greater homing and tissue retention of the gammadelta T-cells when infused into SCID mouse hosts.

Since gammadelta T-cells participate in immune responses during tissue homeostasis, infectious and autoimmune disease, inflammation, transplantation and tumor surveillance, exhibit spontaneous non-MHC-restricted cytotoxic activity against infected and tumor cells, and mediate resistance to viral infections and cancer development in vivo, methods for effectively increasing gammadelta T-cell functionality can be useful for treatment of tumors and elimination of infected cells with stronger response and fewer adverse effects.

Thus, developing protocols for effectively enhancing cultured gammadelta T-cell function and likelihood of their homing and retention in host tissues in-vivo following infusion, could improve the success of therapies, such as adoptive immunotherapy, with gammadelta T-cells for the treatment of solid tumors, malignancies, viral and autoimmune disorders and the like.

Thus, according to one aspect of an embodiment of the present invention there is provided a method of enhancing gammadelta T-cell homing and/or retention potential, the method comprising obtaining a selected cell population enriched for gammadelta T-cells, ex-vivo providing the selected cell population with conditions for gammadelta T-cell expansion, and providing nicotinamide in the range of 0.5 to 50 mM for a period of time sufficient for enhancing gammadelta T-cell homing and/or retention potential, thereby enhancing the homing and/or retention potential of gammadelta T-cells in the selected cell population.

As used herein, the term “gammadelta T-cell” may also be referred to herein as a “γδ T-cell”, a “gammadelta T cell”, or further as a “gd T-cell” or “gd T cell”.

Gammadelta T-cells are defined by expression of heterodimeric T-cell receptors (TCRs) composed of γ (gamma) and δ (delta) chains. This sets them apart from the classical and much better known CD4+ helper T cells and CD8+ cytotoxic T cells that express αβ TCRs. The mechanism of (thymic) selection of γδ T cells is still largely unknown.

Gammadelta T-cells often show tissue-specific localisation of oligoclonal subpopulations sharing the same TCR chains. For instance, human peripheral blood gammadelta-T cells are largely Vgamma9/Vdelta2+, and murine skin gammadelta T cells, so-called dendritic epidermal T cells (DECT cells), are largely Vgamma5/Vdelta1+. In general, gammadelta T-cells are enriched in epithelial and mucosal tissues where they are thought to serve as the first line of defense against pathogenic challenge.

As used herein, “gammadelta T-cell activation” refers to any measurable biological phenomenon associated with a gammadelta T-cell that is representative of such T cell being activated. Non-limiting examples of such a biological phenomenon include an increase of cytokine production, changes in the qualitative or quantitative composition of cell surface proteins, an increase in T cell proliferation, and/or an increase in T cell effector function, such killing of a target cell or assisting another effector cell to kill a target cell.

According to some embodiments of the present invention, the method of the invention enhances homing and/or retention potential of the gammadelta T-cells.

As used herein, the term “function” or “gammadelta T-cell function” refers to any biological function ascribed to gammadelta T-cells. A non-limiting list of gammadelta T-cell functions includes, for example, cytotoxicity, induction of apoptosis, cell motility, directed migration, cytokine and other cell signal response, cytokine/chemokine production and secretion, expression of activating and inhibitory cell surface molecules in-vitro, cell homing and in-vivo retention in a transplanted host, and alteration of disease or disease processes in vivo. In some embodiments, gammadelta T-cell functions enhanced by exposure to nicotinamide and/or other nicotinamide moiety include at least one of elevated expression of CD62L surface marker, elevated migration response, and greater cytotoxic activity of the gammadelta T-cells, as well as elevated homing and in-vivo retention of infused gammadelta T-cells. In specific embodiments, gammadelta T-cell functions enhanced by exposure to nicotinamide and/or other nicotinamide moiety include at least one of elevated expression of CD62L surface marker of the gammadelta T-cells and elevated homing and in-vivo retention of infused gammadelta T-cells. In particular embodiments, both expression of CD62L surface marker of the gammadelta T-cells and homing and in-vivo retention of infused gammadelta T-cells are enhanced by exposure of the gammadelta T-cells to nicotinamide and/or other nicotinamide moiety.

Assays for adhesion and migration molecules such as CD62L, CXCR-4, CD49e and the like, important for homing and retention of cells in transplantation, are well known in the art. CD62L expression in a cell can be assayed, for example, by flow cytometry, immunodetection, quantitative cDNA amplification, hybridization and the like. In one embodiment, CD62L expression is detected in different populations of gammadelta T-cells by exposure of the cells to a fluorescent-tagged specific anti-human CD62L monoclonal antibody [e.g., CD62L PE, Cat. No. 304806 from BioLegend (San Diego, Calif., USA)], and sorting of the cells by fluorescent activated cell sorting (FACS).

Assays for cells migration are well known in the art. Migration of cells can be assayed, for example, by transmigration assays or gap closure assays. In transmigration assays, such as the two-chamber technique, cells are separated from a stimulus by a barrier (e.g., filter), and migration of the cells is detected by counting loss of cells from the origin, accumulation of cells across the barrier, or both, at specific intervals. In the gap closure assay, cells are placed on the periphery of a visible gap (scored agar plate, around a circle, etc.) and incubated with a stimulus. Closure of the space between the cells applied by cell motility, in response to a stimulus, is visualized using cytometry, immunodetection, microscopy/morphometrics, etc. In one embodiment, migration potential of different populations of cells is determined by the “Transwell”™ transmigration assay.

Assays for homing and in-vivo retention of transfused or transplanted cells are well known in the art. As used herein, the term “homing” refers to the ability of a transfused or transplanted cell to reach, and survive, in a host target organ. For example, gammadelta T-cell target organs can be the lymphoid tissue, hepatocytes target organs can be liver parenchyma, alveolar cells target organs can be lung parenchyma, etc. As used herein, the term “in-vivo retention” refers to the ability of the transfused or transplanted cells to populate, optionally proliferate and remain viable in the target organs. Animal models for assaying homing and in-vivo retention of transplanted gammadelta T-cells include, but are not limited to immunodeficient small mammals (such as SCID and IL2Rγ^{null} mice and the like). The SCID-Hu mouse model employs C.B-17 scid/scid (SCID) mice transplanted with human fetal thymus and liver tissue or fetal BM tissue and provides an appropriate model for the evaluation of transplanted human gammadelta T-cells retention and therapeutic potential. Homing and in-vivo retention of transplanted cells can be assessed in human host subjects as well. In one embodiment, homing and in-vivo retention is assayed in irradiated NOD/SCID mice, transfused with, for example, about 15×10⁴, about 15×10⁵, about 15×10⁶, about 15×10⁷ or more human gammadelta T-cell enriched cells cultured with an effective concentrations of nicotinamide according to the present invention, and sacrificed at a predetermined time post transfusion (for example, about 5 hours, 10 hours, 12 hours, 1, 2, 3, 4, 5, 6, 7 days, 1, 2, 3, 4, 5 weeks, 2, 3, 4 months or more post transfusion). Upon sacrifice of the mice, samples of spleen, bone marrow, peripheral blood, and other organs are evaluated by FACS for the presence of human gammadelta T-cells.

Further, the phrase “homing and/or retention potential” refers to the ability of cells (e.g. gammadelta T-cells), when infused into a host organism (e.g. subject), most commonly into the circulation as an intravenous infusion, to exit the circulatory system and populate a host organ or tissue. In particular, the term “retention”, as used herein, refers to the ability of infused cells to remain in a host tissue or organ following “homing” and population of that tissue or organ. As used herein, the phrase “enhancing homing and/or retention potential” refers to an improvement in efficiency, quality or rapidity of cell transplantation which may result from improved homing and/or retention to the target tissue or organ, improved adhesion, reduced rejection and the like.

Assays for cytotoxicity (“cell killing”) are well known in the art. Examples of suitable target cells for use in redirected killing assays are cancer cell line, primary cancer cells solid tumor cells, leukaemic cells, or virally infected cells. Particularly, K562, BL-2, colo250 and primary leukaemic cells can be used, but any of a number of other cell types can be used and are well known in the art (see, e.g., Sivori et al. (1997) J. Exp. Med. 186: 1129-1136; Vitale et al. (1998) J. Exp. Med. 187: 2065-2072; Pessino et al. (1998) J. Exp. Med. 188: 953-960; Neri et al. (2001) Clin. Diag. Lab. Immun. 8:1131-1135). Cell killing is assessed by cell viability assays (e.g., dye exclusion, chromium release, CFSE), metabolic assays (e.g., tetrazolium salts), and direct observation.

Homing and/or retention potential of cells can be determined ex-vivo by measurement of markers of cell functionality (e.g. adhesion molecules such as CD62L, selectin ligand, etc.), or by in-vivo infusion and transplantation in the SCID-Hu mouse model. The SCID-Hu mouse model employs C.B-17 scid/scid (SCID) mice transplanted with human fetal thymus and liver tissue or fetal BM tissue and provides an appropriate model for the evaluation of transplantable putative human lymphoid and other cells. Because of the reconstitution of the SCID mice with human fetal tissue, the model affords the homing and retention of human cells and function in a microenvironment of human origin. Mice are typically irradiated, then delivered lymphoid cells into the grafts, and homing/retention is measured by any number of methods, including FACS and immunohistochemistry of repopulated organs (for example, see Materials and Experimental Methods below).

As used herein the term “ex-vivo” refers to a process in which cells are removed from a living organism and are propagated outside the organism (e.g., in a test tube).

As used herein, the term “in-vitro” refers to a process in which cells originating from a cell line or lines (such as NTera2 neural cells, embryonic cell lines, etc.) maintained in the laboratory, are manipulated outside of an organism. Such cell lines are often immortalized cells.

As used herein the phrase “population of cells” refers to a homogeneous or heterogeneous isolated population of cells which can comprise cell populations suitable for expansion or transplantation according to the methods of the invention. In a preferred embodiment, at least a portion of the population of cells of this aspect of the present invention are gammadelta T-cells, expressing heterodimeric TCRs comprising γ (gamma) and δ (delta) chains on the cell-surface.

In some aspects, the present disclosure provides methods for the ex vivo expansion of a population of gammadelta T-cells. A gammadelta T-cell or gammadelta T-cell population of the disclosure may be expanded ex vivo. A gammadelta T-cell or gammadelta T-cell population of the disclosure can be expanded without activation by APCs, or without co-culture with APCs and aminophosphates.

According to some embodiments of the method of the invention, the gammadelta T-cells are provided with conditions for gammadelta T-cell expansion.

In specific embodiments, the conditions for gammadelta T-cell expansion comprise provision of nutrients and cytokines.

Suitable culture media capable of supporting gammadelta T-cells include HEM, DMEM, RPMI, F-12, and the like. If required, the medium can contain supplements required for cellular metabolism such as glutamine and other amino acids, vitamins, minerals and useful proteins such as transferrin, and the like. The medium may or may not contain added serum. The medium may also contain antibiotics to prevent contamination with yeast, bacteria, and fungi, such as penicillin, streptomycin, gentamicin, and the like. If cells are to be cultured, conditions should be close to physiological conditions (preferably, a pH of about 6 to about 8, and a temperature of about 30° C. to about 40° C.). In some embodiments, the culture medium can be optionally supplemented with at least one proliferation-inducing growth factors, cytokines and/or chemokines such as IL-2, IL, IL-4, IL-7, IL-15, IL-12, IL-21, IL-23 or IL-33 and combinations thereof. In specific embodiments, the culture medium is supplemented with IL-2, and/or IL-15. In addition to proliferation-inducing growth factors, other growth factors may be added to the culture medium. In some exemplary embodiments, gammadelta T-Cells are stimulated and expanded in serum-free media such as Ex-Vivo 10, Ex-Vivo 15, Ex-Vivo 20, AIMV media, Optimizer CTS, containing cytokines (IL-2, IL-4, IL-7, IL-15, IL-12, IL-21, IL-23 or IL-33), growth factors (insulin and transferrin, insulin-like growth factors), albumin, lipids (cholesterol, lipid solutions, lipid pre-cursors), vitamins, copper, iron, selenium, protein hydrolysate, essential amino acids, non-essential amino acids, and shear protectant (Pluronic F-68).

Cytokines and other growth factors are typically provided in concentrations ranging from 0.5-100 ng/ml, or 1.0-80 ng/ml, more typically 5-750 ng/ml, yet more typically 5.0-50 ng/ml (up to 10× such concentrations may be contemplated), and are available commercially, for example, from Perpo Tech, Inc., Rocky Hill, N.J., USA. In one embodiment, conditions allowing for cell proliferation includes providing the cytokine interleukin 2 or interleukin 15. In specific embodiments, the gammadelta T-cells are cultured with 20 ng/ml IL-15 and/or IL-2.

Further, it will be appreciated in this respect that novel cytokines are continuously discovered, some of which may find uses in the methods of gammadelta T-cell proliferation of the present invention. For applications, in which cells are introduced (or reintroduced) into a human subject, it is often preferable to use serum-free formulations, such as AIM V® serum free medium for lymphocyte culture or MARROWMAX® bone marrow medium. Such medium formulations and supplements are available from commercial sources such as Invitrogen (GIBCO) (Carlsbad, Calif.). The cultures can be supplemented with amino acids, antibiotics, and/or with cytokines to promote optimal viability, proliferation, functionality and/or and survival.

Such serum-free media can be further supplemented with additives to support high cell density gammadelta T-cell growth in suspension culture (e.g. WAVE bioreactor) while maintaining biological functionality of the gammadelta T-cells.

Ex-vivo culturing of gammadelta T-cells can be effected, according to one aspect of the present invention, by providing gammadelta T-cells or gammadelta T-cell populations ex vivo with conditions for cell proliferation and ex vivo culturing the gammadelta T-cells with a nicotinamide moiety, thereby ex-vivo expanding and/or ex-vivo enhancing homing and/or retention potential of the population of gammadelta T-cells.

As used herein “culturing” includes providing the chemical and physical conditions (e.g., temperature, gas) which are required for gammadelta T-cell maintenance, and, optionally, growth factors. In one embodiment, culturing the gammadelta T-cells includes providing the gammadelta T-cells with conditions for gammadelta T-cell expansion (e.g. proliferation). Examples of chemical conditions which may support gammadelta T-cell expansion include but are not limited to buffers, nutrients, serum, vitamins and antibiotics as well as cytokines and other growth factors which are typically provided in the growth (i.e., culture) medium. In a particular embodiment, conditions for cell growth comprise nutrients, serum and cytokine(s). In one embodiment, the gammadelta T-culture medium includes a minimal essential medium (MEM), such as MEMα (BI, Bet HaEmek, Israel) and serum. In a particular embodiment, the culture medium is MEMα comprising 10% Human AB Serum (Sigma-Aldrich, St. Louis, Mo.). Other media suitable for use with the invention include, but are not limited to Glascow's medium (Gibco Carlsbad Calif.), RPMI medium (Sigma-Aldrich, St Louis Mo.) or DMEM (Sigma-Aldrich, St Louis Mo.). It will be noted that many of the culture media contain nicotinamide as a vitamin supplement for example, MEMα (8.19 μM nicotinamide), RPMI (8.19 μM nicotinamide), DMEM (32.78 μM nicotinamide) and Glascow's medium (16.39 μM nicotinamide), however, the methods of the present invention relate to exogenously added nicotinamide supplementing any nicotinamide and/or nicotinamide moiety included the medium's formula, or that resulting from overall adjustment of medium component concentrations.

According to one embodiment, the gammadelta T-cell or gammadelta T-cell population is cultured with nutrients, serum, a cytokine(s) (e.g. IL-15 and/or IL-2) and nicotinamide and/or a nicotinamide moiety. As used herein, the term “nicotinamide moiety” refers to nicotinamide as well as to products that are derived from nicotinamide, derivatives, analogs and metabolites thereof, such as, for example, NAD, NADH and NADPH, which are capable of effectively and preferentially enhancing gammadelta T-cell homing and/or retention. Nicotinamide derivatives, analogs and metabolites can be screened and evaluated for their effect on homing and/or retention in culture by addition to gammadelta T-cell cultures maintained as described herein, addition to functional assays such as cell adhesion, rolling and motility assays, or in automated screening protocols for homing and/or retention markers designed for high-throughput assays well known in the art.

As used herein, the phrase “nicotinamide analog” refers to any molecule that is known to act similarly to nicotinamide in the abovementioned or similar assays. Representative examples of nicotinamide analogs can include, without limitation, benzamide, nicotinethioamide (the thiol analog of nicotinamide), nicotinic acid and α-amino-3-indolepropionic acid.

The phrase “nicotinamide derivative” further refers to any structural derivative of nicotinamide itself or of an analog of nicotinamide. Examples of such derivatives include, without limitation, substituted benzamides, substituted nicotinamides and nicotinethioamides and N-substituted nicotinamides and nicotinthioamides, 3-acetylpiridine and sodium nicotinate. In one particular embodiment of the invention the nicotinamide moiety is nicotinamide.

Nicotinamide or nicotinamide moiety concentrations suitable for use in some embodiments of the present invention are typically in the range of about 0.5 mM to about 50 mM, about 1.0 mM to about 25 mM, about 1.0 mM to about 25 mM, about 2.5 mM to about 10 mM, about 5.0 mM to about 10 mM, about 0.5 mM to 20 mM. Exemplary effective concentrations of nicotinamide can be of about 0.5 to about 15 mM, 1.0-10.0 mM, typically 2.5 or 5.0 mM, based on the effect of these concentrations of nicotinamide on homing and/or retention of gammadelta T-cells. According to some embodiments of the invention, nicotinamide is provided at a concentration in the range (mM) of about 0.5, about 0.75, about 1.0, about 1.25, about 1.5, about 1.75, about 2.0, about 2.25, about 2.5, about 2.75, about 3.0, about 3.25, about 3.5, about 3.75, about 4.0, about 4.25, about 4.5, about 4.75, about 5.0, about 5.25, about 5.5, about 5.75, about 6.0, about 6.25, about 6.5, about 6.75, about 7.0, about 7.25, about 7.5, about 7.75, about 8.0, about 8.25, about 8.5, about 8.75, about 9.0, about 9.25, about 9.5, about 9.75, about 10.0, about 11.0, about 12.0, about 13.0, about 14.0, about 15.0, about 16.0, about 17.0, about 18.0, about 20.0 mM, about 23.0 mM, about 25.0 mM, about 30.0 mM, about 35.0 mM, about 40.0 mM, about 45.0 mM or about 50.0 mM. All effective intermediate concentrations are contemplated. In specific embodiments, conditions allowing proliferation comprise between 0.5 to 50 mM, 1.0 to 10.0 mM nicotinamide. In yet other embodiments, conditions enhancing homing and/or retention of gammadelta T-cells comprise 5.0 mM nicotinamide.

Suitable concentrations of the nicotinamide and/or nicotinamide moiety can be determined according to any assay of gammadelta T-cell homing and/or retention, or CD62L expression. Suitable concentration of nicotinamide is a concentration which use thereof in culture “enhances”, or results in a net increase of function of gammadelta T-cell homing and/or retention, compared to “control” cultures having less than 0.1 mM, less than 0.2 mM, or less than 0.4 mM of the nicotinamide and tested from the same gammadelta T-cell source (e.g. cord blood, bone marrow or peripheral blood preparation), in the same assay and under similar culture conditions (duration of exposure to nicotinamide, time of exposure to nicotinamide, conditions for expansion).

It will be noted that conditions for expansion and enhancement of gammadelta T-cells according to the methods of the present invention may also be favorable for culture of other types of cells found in a mixed population of cells with gammadelta T-cells. Thus, in some embodiments, providing the conditions for gammadelta T-cell expansion and nicotinamide according to the methods disclosed herein also enhances homing and/or retention potential of other lymphoid cells, for example, NK cells, providing expanded cell populations of potentially greater therapeutic efficacy than similar cell populations cultured and/or expanded without additional nicotinamide.

In specific embodiments the method of the invention can expand and enhance functionality of various gammadelta T-cell(s) populations, such as a Vgamma1+, a Vgamma2+, Vgamma3+, gammadelta T-cell population. In some instances, a gammadelta T-cell population can be cultured ex-vivo in fewer than 36 days, fewer than 35 days, fewer than 34 days, fewer than 33 days, fewer than 32 days, fewer than 31 days, fewer than 30 days, fewer than 29 days, fewer than 28 days, fewer than 27 days, fewer than 26 days, fewer than 25 days, fewer than 24 days, fewer than 23 days, fewer than 22 days, fewer than 21 days, fewer than 20 days, fewer than 19 days, fewer than 18 days, fewer than 17 days, fewer than 16 days, fewer than 15 days, fewer than 14 days, fewer than 13 days, fewer than 12 days, fewer than 11 days, fewer than 10 days, fewer than 9 days, fewer than 8 days, fewer than 7 days, fewer than 6 days, fewer than 5 days, fewer than 4 days, fewer than 3 days. In some instances the gammadelta T-cell population is cultured for between 1 and 8 weeks, between 1 and 5 weeks, between 1 and 4 weeks, between 1 and 3 weeks, between 1 and 2 weeks, between 1 and 14 days, between 2 and 13 days, between 1 and 10 days, between 2 and 8 days, between 1 and 7 days, between 3 and 12 days and between 5 and 14 days. In some embodiments short-term ex-vivo exposure of gammadelta T-cell enriched cells to nicotinamide and/or other nicotinamide moiety, for periods of minutes, hours, 1 day, and the like is also envisaged.

In some studies, ex-vivo expansion of gammadelta T-cells by culture with nutrients, serum, cytokines and nicotinamide does not require replenishing the medium or manipulation over the culture period, while other studies have advocated culture medium replenishment (“re-feeding”) at different intervals during the gammadelta T-cell culture. In certain embodiments of the present invention, the gammadelta T-cell fraction is “re-fed” during the culture period. Thus, in specific embodiments, culturing the gammadelta T-cell population comprises supplementing the gammadelta T-cell enriched cells with fresh nutrients, serum, cytokines and nicotinamide 8-10 days following initiation of the ex-vivo culture. In some embodiments, supplementing is provided between 8-9 days following initiation of the ex-vivo culture, between 9-10 days following initiation of the ex-vivo culture, or between 8-10 days following initiation of culturing of the gammadelta T-cell enriched cells. In some embodiments, supplementing (or “refeeding”) comprises removing about 30-80%, about 40-70% or about 45-55% of the medium of the culture, and replacing that with a similar (e.g. equivalent) volume of fresh medium having the same composition and level of nutrients, serum, cytokines (e.g. IL-2 and/or IL-15) and nicotinamide as the removed medium. In some embodiments, supplementing (or “refeeding”) comprises removing about 50% of the medium of the culture, and replacing the removed medium with a similar (e.g. equivalent) volume of fresh medium having the same composition and level of nutrients, serum, cytokines (e.g. IL-2 and/or IL-15) and nicotinamide. In other embodiments, culture volume following refeeding reaches approximately twice the original culture volume at initiation of the gammadelta T-cell enriched cell culture (“seeding”).

Gammadelta cell populations can be cultured using a variety of methods and devices. Selection of culture apparatus is usually based on the scale and purpose of the culture. Scaling up of cell culture preferably involves the use of dedicated devices. In some embodiments, culturing the gammadelta T-cell enriched fractions is effected in flasks, at a cell density of 100-4000×10⁶ cells per flask. In specific embodiments, culturing the gammadelta T-cell enriched fractions (e.g. initiation of the ex-vivo culture and/or “re-feeding”) is effected in flasks, at a cell density of 200-300×10⁶ cells per flask. In certain embodiments, the flasks are flasks comprising a gas-permeable membrane, such as the G-Rex culture device (G-Rex 100M or closed system G-Rex MCS, WolfWilson, St Paul Minn.).

Culturing the gammadelta T-cell enriched cells can be effected with or without feeder cells or a feeder cell layer. Feeder layer-free ex-vivo culture is highly advantageous for clinical applications of cultured cells. Thus, according to one embodiment, culturing the population of gammadelta T-cell enriched cells is effected without feeder layer or feeder cells.

In some aspects, provided are methods for expanding various gammadelta T-cells or gammadelta T-cell populations, by contacting the gammadelta T-cells with an activation agent. In some cases, the activation agent binds to a specific epitope on a cell-surface receptor of a gammadelta T-cell, such as a monoclonal antibody. The activation agent can specifically activate the growth of one or more types of gammadelta T-cells, such delta1, delta2, or delta3 cell populations. In some embodiments the activation agent specifically activates the growth of delta1 cell populations. In other cases, the activation agent specifically activates the growth of delta2 cell populations. An activation agent may stimulate the expansion of gammadelta T-cells at a fast rate of growth.

In some embodiments, the gammadelta T-cell population comprises different percentages of delta1, delta2, and delta3 T-cells. A gammadelta T-cell population can comprise, for example, fewer than 90% delta1 T-cells, or delta2 T-cells, or delta3 T-cells, fewer than 80% delta1 T-cells, or delta2 T-cells, or delta3 T-cells, fewer than 70% delta1 T-cells, or delta2 T-cells, or delta3 T-cells, fewer than 60% delta1 T-cells, or delta2 T-cells, or delta3 T-cells, fewer than 50% delta1 T-cells, or delta2 T-cells, or delta3 T-cells, fewer than 40% delta1 T-cells, or delta2 T-cells, or delta3 T-cells, fewer than 30% delta1 T-cells, or delta2 T-cells, or delta3 T-cells, fewer than 20% delta1 T-cells, or delta2 T-cells, or delta3 T-cells, fewer than 10% delta1 T-cells, or delta2 T-cells, or delta3 T-cells, or fewer than 5% delta1 T-cells, or delta2 T-cells, or delta3 T-cells. Alternatively, a gammadelta T-cell population can comprise greater than 5% delta1 T-cells, or delta2 T-cells, or delta3 T-cells, greater than 10% delta1 T-cells, or delta2 T-cells, or delta3 T-cells, greater than 20% delta1 T-cells, or delta2 T-cells, or delta3 T-cells, greater than 30% delta1 T-cells, or delta2 T-cells, or delta3 T-cells, greater than 40% delta1 T-cells, or delta2 T-cells, or delta3 T-cells, greater than 50% delta1 T-cells, or delta2 T-cells, or delta3 T-cells, greater than 60% delta1 T-cells, or delta2 T-cells, or delta3 T-cells, greater than 70% delta1 T-cells, or delta2 T-cells, or delta3 T-cells, greater than 80% delta1 T-cells, or delta2 T-cells, or delta3 T-cells, or greater than 90% delta1 T-cells, or delta2 T-cells, or delta3 T-cells.

In some embodiments, gammadelta T-cell(s) can rapidly expand in response to contact with one or more antigens. Some gammadelta T-cell(s), such as Vgamma9Vdelta2+ gammadelta T-cell(s) rapidly expand ex vivo in response to contact with some antigens, like prenyl-pyrophosphates, alkyl amines, and metabolites or microbial extracts during tissue culture. In addition, some wild-type gammadelta T-cell(s), such as Vgamma2Vdelta2+ gammadelta T-cell(s) rapidly expand in vivo in humans in response to certain types of vaccination(s). Stimulated gammadelta T-cells can exhibit numerous antigen-presentation, co-stimulation, and adhesion molecules that can facilitate the isolation of a gammadelta T-cell(s) from a complex sample. A gammadelta T-cell(s) within a complex sample can be stimulated in vitro with at least one antigen for 1 day, 2 days, 3 days, 4 days, 5 days, 6 days, 7 days, or another suitable period of time. Stimulation of the gammadelta T-cell with a suitable antigen can ex-vivo expand the gammadelta T-cell population.

Non-limiting examples of antigens that may be used to stimulate the expansion of gammadelta T-cell(s) from a complex sample include prenyl-pyrophosphates, such as isopentenyl pyrophosphate (IPP), alkyl-amines, metabolites of human microbial pathogens, metabolites of commensal bacteria, -methyl-3-butenyl-1-pyrophosphate (2M3B1PP), (E)-4-hydroxy-3-methyl-but-2-enyl pyrophosphate (HMB-PP), ethyl pyrophosphate (EPP), farnesyl pyrophosphate (FPP), dimethylallyl phosphate (DMAP), dimethylallyl pyrophosphate (DMAPP), ethyl-adenosine triphosphate (EPPPA), geranyl pyrophosphate (GPP), geranylgeranyl pyrophosphate (GGPP), isopentenyl-adenosine triphosphate (IPPPA), monoethyl phosphate (MEP), monoethyl pyrophosphate (MEPP), 3-formyl-1-butyl-pyrophosphate (TUBAg 1), X-pyrophosphate (TUBAg 2), 3-formyl-1-butyl-uridine triphosphate (TUBAg 3), 3-formyl-1-butyl-deoxythymidine triphosphate (TUBAg 4), monoethyl alkylamines, allyl pyrophosphate, crotoyl pyrophosphate, dimethylallyl-gamma-uridine triphosphate, crotoyl-gamma-uridine triphosphate, allyl-gamma-uridine triphosphate, ethylamine, isobutylamine, sec-butylamine, iso-amylamine and nitrogen containing bisphosphonates.

Activation and/or expansion of gammadelta T-cells can be performed using activation and co-stimulatory agents described herein to trigger specific gammadelta T-cell proliferation and persistence populations. In some embodiments, activation and expansion of gammadelta T-cells from different cultures can achieve distinct clonal or mixed polyclonal population subsets. In some embodiments, different agonist agents can be used to identify agents that provide specific gammadelta activating signals. In one aspect, agents that provide specific gammadelta activating signals can be different monoclonal antibodies (MAbs) directed against the gammadelta TCRs. In one aspect, the MAbs can bind to different epitopes on the constant or variable regions of gamma TCR and/or delta TCR. In one aspect, the MAbs can include gammadelta TCR pan MAbs. In one aspect, the gammadelta TCR pan MAbs may recognize domains shared by different gamma and delta TCRs on both, including delta1 and delta2 cell populations. In one aspect, the antibodies may be 5A6.E9 (Thermo scientific), B1 (Biolegend), IMMU510 and/or 11F12 (Beckman Coulter). In one aspect, the MAbs can be directed to specific domains unique to the variable regions of the gamma chain (7A5 Mab, directed to like Vgamma9 TCR (Thermo Scientific #TCR1720)), or domains on Vdelta1 variable region (Mab TS8.2 (Thermo scientific #TCR1730; MAb TC1, MAb R9.12 (Beckman Coulter)), or Vdelta2 chain (MAb 15D (Thermo Scientific #TCR1732)). In some embodiments, antibodies against different domains of the gammadelta TCR (pan antibodies and antibodies recognizing specific variable region epitopes on subset populations) can be combined. In some embodiments, gammadelta T-cells activators can include gammadelta TCR-binding agents such as MICA, agonist antibody to NKG2D, (Fc tag) fusion protein of MICA, ULBP1, ULBP3 (R&D systems Minneapolis, Minn.) ULBP2, or ULBP6 (Sino Biological Beijing, China). In some embodiments, companion co-stimulatory agents to assist in triggering specific gammadelta T cell proliferation without induction of cell anergy and apoptosis can be used in combination, such as, but not limited to ligands to receptors expressed on gammadelta cells, such as NKG2D, CD161, CD70, JAML, DNAX accessory molecule-1 (DNAM-1) ICOS, CD27, CD137, CD30, HVEM, SLAM, CD122, DAP, and CD28- or antibodies specific to unique epitopes on CD2 and CD3 molecules.

Non-limiting example of reagents that can be used to facilitate the expansion of a gammadelta T-cell population ex-vivo include anti-CD3 or anti-CD2, anti-CD27, anti-CD30, anti-CD70, anti-OX40 antibodies, IL-2, IL-15, IL-12, IL-9, IL-33, IL-18, or IL-21, CD70 (CD27 ligand), phytohaemagglutinin (PHA), concavalin A (ConA), pokeweed (PWM), protein peanut agglutinin (PNA), soybean agglutinin (SBA), Les Culinaris Agglutinin (LCA), Pisum Sativum Agglutinin (PSA), Helix pomatia agglutinin (HPA), Vicia graminea Lectin (VGA), or another suitable mitogen capable of stimulating T-cell proliferation.

In some aspects, the present disclosure provides methods for the culturing of gammadelta T-cells that have been isolated from a subject. A gammadelta T-cell can be isolated from a complex sample of a subject. A complex sample can be a peripheral blood sample, a cord blood sample, a tumor, a stem cell precursor, a tumor biopsy, a tissue, a lymph, or from epithelial sites of a subject directly contacting the external milieu, or derived from stem precursor cells. In particular embodiments, the sample is derived from an organ selected from the group consisting of a muscle, skin, bone, lymph organ, pancreas, liver, gallbladder, kidney, digestive tract organ, respiratory tract organ, reproductive organ, urinary tract organ, a blood-associated organ, a thymus, a spleen and a nervous system organ. In specific embodiments, the gammadelta cells or gammadelta cell population is derived from a source selected from the group consisting of hematopoietic cells, umbilical cord cells, peripheral blood cells (mobilized or not mobilized) and bone marrow cells. In yet further embodiments, gammadelta cells or populations are isolated from bone marrow or peripheral blood samples, neonatal umbilical cord blood, or from a mononuclear cell fraction.

A gammadelta T-cell may be directly isolated from a complex sample of a subject, for example, by sorting gammadelta T-cell(s) that express one or more cell surface markers with flow cytometry techniques. Wild-type gammadelta T-cells exhibit numerous antigen recognition, antigen-presentation, co-stimulation, and adhesion molecules that can be associated with a gammadelta T-cell(s). One or more cell surface markers such as specific gammadelta TCRs, antigen recognition, antigen-presentation, ligands, adhesion molecules, or co-stimulatory molecules may be used to isolate a wild-type gammadelta T-cell from a complex sample. Various molecules associated with, or expressed by, a gammadelta T-cell may be used to isolate a gammadelta T-cell from a complex sample. In some embodiments, the present disclosure provides methods for isolation of mixed population of Vdelta1+, Vdelta2+, Vdelta3+ cells or any combination thereof.

Peripheral blood mononuclear cells can be collected from a subject, for example, with an apheresis machine, including the Ficoll-Paque™ PLUS (GE Healthcare) system, or another suitable device/system. Gammadelta T-cell(s), or a desired subpopulation of gammadelta T-cell(s), can be purified from the collected sample with, for example, with flow cytometry techniques. Cord blood cells can also be obtained from cord blood during the birth of a subject. In particular embodiments, the gammadelta T-cell or gammadelta T-cell population is from an apheresis sample, or derived from an apheresis sample.

Positive and/or negative selection of cell surface markers expressed on the collected gammadelta T-cell(s) can be used to directly isolate a gammadelta T-cell, or a population of gammadelta T-cell(s) expressing similar cell surface markers from a peripheral blood sample, a cord blood sample, a tumor, a tumor biopsy, a tissue, a lymph, or from an epithelial sample of a subject. For instance, a gammadelta T-cell can be isolated from a complex sample based on positive or negative expression of CD2, CD3, CD4, CD8, CD24, CD25, CD44, Kit, TCRalpha, TCRbeta, TCRdelta, NKG2D, CD70, CD27, CD30, CD16, CD337 (NKp30), CD336 (NKp46), OX40, CD46, CCR7, and other suitable cell surface markers. In particular embodiments, the selected cell population is a lymphocyte cell population enriched for gammadelta T-cells by TCRalphabeta T-cell depletion (negative selection). It will be appreciated that such a gammadelta T-cell enriched cell population can include significant fractions of other cell types, such as NK cells.

In still other embodiments, the selected cell population is a lymphocyte cell population enriched for gammadelta T-cells by positive TCRgammadelta T-cell selection. It will be appreciated that non-gammadelta T-cells will be scarce in such a gammadelta T-cell enriched cell population. In some embodiments, the gammadelta T-cell positive selected enriched cell population is devoid of NK cells.

A gammadelta T-cell may be isolated from a complex sample that is cultured ex-vivo. In specific embodiments, enriched gammadelta T-cell populations can be generated prior to their specific activation and expansion. In some embodiments, additional cell populations such as monocytes, T-cells, B-cells, and NK cells are included in the enriched gammadelta T-cell population, and in some cases can be activated and expanded along with the gammadelta T-cells. In some aspects, activation and expansion of gammadelta T-cells are performed without the presence of native or engineered APCs. In some aspects, isolation and expansion of gammadelta T cells from tumor specimens can be performed using immobilized gammadelta T cell mitogens, including antibodies specific to gammadelta TCR, and other gammadelta TCR activating agents, including lectins.

In certain embodiments, the gammadelta T-cell enriched cells are harvested from the culture 14-16 days following initiation of the gammadelta T-cell enriched cell culture. Harvesting of the cells can be performed manually, by releasing attached cells (e.g. “scraping” culture vessel surfaces) or by a cell harvesting device, which is designed to efficiently wash cells out of their culture vessels and collect the cells automatically. In specific embodiments, the expanded cell fraction is harvested from the culture vessels by a cell harvesting device (e.g. the harvesting device of the G-Rex MCS, WolfWilson, St Paul Minn.).

In some embodiments, harvesting of expanded gammadelta T-cell enriched cells from culture removes most, or nearly all of the cells from the culture vessel. In other embodiments, harvesting can be performed in two or more steps, allowing the unharvested cells to remain in culture until harvested at a later time. Harvesting the two portions can be performed with an interval of hours, days or more between harvesting of the first and second portion.

In order to prepare the expanded gammadelta T-cell enriched cells for transplantation, the harvested cells need to be washed of culture medium, critical parameters evaluated and volume adjusted to a concentration suitable for infusion over a clinically reasonable period of time.

Following harvesting, the expanded gammadelta T-cell enriched cell population can be washed free of culture medium manually or, preferably for clinical applications, using an automated device employing a closed system. Washed cells can be reconstituted with an infusion solution (for example, one exemplary infusion solution comprises 8% w/v HSA and 6.8% w/v Dextran-40). In some embodiments, the reconstitution is performed in a closed system. In some embodiments, the infusion solution is screened for suitability for use with the methods and compositions of the present invention. Exemplary criteria for selection of suitable infusion solution include safety tests indicating no bacterial, yeast or mold growth, endotoxin content of less than 0.5 Eu/ml and a clear, foreign particle-free appearance.

The methods described hereinabove for ex-vivo culturing gammadelta T-cells and gammadelta T-cell enriched populations can result, inter alia, in a cultured population of gammadelta T-cells and gammadelta T-cell enriched cells.

Thus, further according to an aspect of the present invention there is provided a population of gammadelta T-cells characterized by at least one of elevated expression of CD62L, elevated migration response, elevated homing and in-vivo retention and increased cytotoxic activity as compared to a population of gammadelta T-cells and/or gammadelta T-cell enriched cells cultured under otherwise identical culturing conditions with less than 0.1 mM of the nicotinamide and/or other nicotinamide moiety. In some embodiments, the population of gammadelta T-cells and/or gammadelta T-cell enriched cells is characterized by at least any two, at least any three, at least any four or all five of elevated expression of CD2L, elevated migration response, elevated homing and/or in-vivo retention, and increased cytotoxic activity, as compared to a population of gammadelta T-cell enriched cells cultured under otherwise identical culturing conditions with less than 0.1 mM of the nicotinamide and/or other nicotinamide moiety.

In Example 1, the inventors have shown that gammadelta T-cell populations prepared according to the methods of the invention have increased expression of cell surface parker CD62L (L-selectin), important to cell adhesion and “rolling”. In Example 2 the inventors have shown that gammadelta T-cell enriched populations prepared according to the methods of the invention have increased in-vivo functional potential, as demonstrated by localization and in-vivo retention in the target organs (e.g., spleen, bone marrow). Thus, in a particular aspect of some embodiments of the present invention there is provided a population of gammadelta T-cell enriched cells characterized by at least one of enhanced CD62L expression and enhanced homing and/or in-vivo retention when transplanted.

In some embodiments, the gammadelta T-cells can be genetically engineered. Genetic engineering of the gammadelta T-cell(s) may comprise stably integrating a construct expressing a tumor recognition moiety, such as an alphabeta TCR, a gammadelta TCR, a CAR encoding an antibody, an antigen binding fragment thereof, or a lymphocyte activation domain into the genome of the isolated gammadelta T-cell(s), a cytokine (e.g. IL-15, IL-12, IL-2, IL-7, IL-21, IL-18, IL-19, IL-33, IL-4, IL-9, IL-23, IL1beta) to enhance T-cell proliferation, survival, and function ex vivo and in vivo. Genetic engineering of the isolated gammadelta T-cell may also comprise deleting or disrupting gene expression from one or more endogenous genes in the genome the isolated gammadelta T-cell, such as the MHC locus (loci).

Gene therapy: For successful long-term gene therapy, a high frequency of genetically modified cells with transgenes stably integrated within their genome, is an obligatory requirement. Viral-based (e.g., retroviral) vectors require active cell division for integration of the transgene into the host genome. Therefore, gene transfer into some fresh cell populations, being unstimulated, is highly inefficient. The ability to store and process a selected population of gammadelta T-cells ex-vivo, and enhance their homing and retention potential would provide for an increased probability of the successful use of genetically modified cell transplantation.

Adoptive immunotherapy: Ex-vivo-expanded, defined lymphoid subpopulations have been studied and used for adoptive immunotherapy of various malignancies, immunodeficiencies, viral and genetic diseases [Freedman Nature Medicine 2: 46, (1996); Heslop Nature Medicine 2: 551, (1996); Protti Cancer Res 56: 1210, (1996)].

The treatment enhances the required immune response or replaces deficient functions. This approach was pioneered clinically by Rosenberg et al. [Rosenberg J Natl Cancer Inst. 85: 622, 1993] using a large number of autologous and also allogeneic ex-vivo expanded non-specific killer T cells, and subsequently ex-vivo expanded specific tumor infiltrating lymphocytes.

As detailed herein, gammadelta T-cells are highly desirable for therapeutic applications. Thus, in one aspect of an embodiment of the invention there is provided a therapeutic cell composition comprising an expanded selected gammadelta T-cell population, the cell population ex-vivo cultured with conditions for gammadelta T-cell expansion and amount of nicotinamide in the range of 0.5-50 mM, wherein the expanded selected gammadelta T-cell population is characterized by at least one of enhanced gammadelta T-cell homing and/or retention potential and enhanced expression of cell surface marker CD62L (L-selectin), as compared to a similar selected gammadelta T-cell population expanded with identical conditions and no more than 0.1 mM nicotinamide. In particular embodiments, the therapeutic cell composition comprises gammadelta T-cells cultured according to the methods of the invention.

In some embodiments, the therapeutic cell composition is a pharmaceutical composition comprising an expanded gammadelta T-cell enriched population, and a pharmaceutically acceptable carrier. As is discussed in detail hereinabove, ex-vivo culture of gammadelta T-cell enriched cells can be advantageously utilized in gammadelta T-cell transplantation or implantation. Hence, according to another aspect of the present invention there is provided a method of gammadelta T-cells or gammadelta T-cell enriched cells or population transplantation or implantation into a recipient. The method according to this aspect of the present invention is effected by (a) ex-vivo expanding a selected gammadelta T-cell population by culturing said cell population conditions for gammadelta T-cell expansion and nicotinamide according to the methods of the present invention, wherein said expanded selected gammadelta T-cell population has enhanced gammadelta T-cell homing and/or retention potential, as compared to a similar selected gammadelta T-cell population expanded without 0.5-50 nM nicotinamide, and (b) infusing the expanded gammadelta T-cells into a subject in need thereof.

The present invention also envisages pharmaceutical compositions comprising expanded gammadelta T-cells or population(s) prepared according to the methods of the invention, and a pharmaceutically acceptable carrier. Expanded gammadelta T-cells or population(s) prepared according to the methods of the invention, and pharmaceutical compositions containing an expanded gammadelta T-cell population described herein may be administered for prophylactic and/or therapeutic treatments. In therapeutic applications, the compositions can be administered to a subject already suffering from a disease or condition in an amount sufficient to cure or at least partially arrest the symptoms of the disease or condition. A gammadelta T-cell or population can also be administered to lessen a likelihood of developing, contracting, or worsening a condition. Effective amounts of a population of expanded gammadelta T-cells, or compositions comprising gammadelta T-cells expanded according to the methods of the present invention for therapeutic use can vary based on the severity and course of the disease or condition, previous therapy, the subject's health status, weight, and/or response to the drugs, and/or the judgment of the treating physician.

An expanded gammadelta T-cell population or composition comprising such of the disclosure can be used to treat a subject in need of treatment for a condition. Examples of conditions include cancer, infectious disease, autoimmune disorder and sepsis. Subjects can be humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In specific embodiments, the subject being treated is a human subject. The subject can be of any age. Subjects can be, for example, elderly adults, adults, adolescents, pre-adolescents, children, toddlers, infants.

A method of treating a condition (e.g., ailment) in a subject with an expanded gammadelta T-cell population, or composition comprising a gammadelta T-cell population expanded according to the methods of the present invention may comprise administering to the subject a therapeutically-effective amount of expanded gammadelta T-cell(s) or of an expanded gammadelta-T-cell population of the invention. A gammadelta T-cell of the disclosure may be administered at various regimens (e.g., timing, concentration, dosage, spacing between treatment, and/or formulation). A subject receiving or to receive administration of such a gammadelta T-cell(s) or gammadelta T-cell population can also be preconditioned with, for example, chemotherapy, radiation, or a combination of both, prior to receiving a gammadelta T-cell or gammadelta T-cell population of the disclosure. As part of a treatment, a gammadelta T-cell or gammadelta T-cell population may be administered to a subject at a first regimen and the subject may be monitored to determine whether the treatment at the first regimen meets a given level of therapeutic efficacy. In some cases, the gammadelta T-cell or gammadelta T-cell population, or another gammadelta T-cell or gammadelta T-cell population may be administered to the subject at a second regimen. In one exemplary a method for treating a subject at least one gammadelta T-cell or gammadelta T-cell population is administered to a subject that has or is suspected of having a given condition (e.g., cancer), optionally administered at a first regimen. Subsequently, the subject may be monitored, for example by a healthcare provider (e.g., treating physician or nurse), for example, to determine or gauge an efficacy of the expanded gammadelta T-cell(s) or gammadelta T-cell population in treating the condition of the subject, or also to determine the in vivo expansion, homing and/or retention of a gammadelta T-cell population in the subject. In some embodiments, at least one other gammadelta T-cell or gammadelta T-cell population is administered to the subject at a second regimen, which may be the same as the first regimen or different than the first regimen. In some situations the administration of the gammadelta T-cell or gammadelta T-cell population is found to be effective (e.g., a single round of administration may be sufficient to treat the condition) and is sufficient. Due to their allogeneic and universal donor characteristics, a population of expanded gammadelta T-cells may be administrated to various subjects, with different MHC haplotypes. A gammadelta T-cell or gammadelta T-cell population may be frozen or cryopreserved prior to being administered to a subject.

In some embodiments, the subject receiving or to receive administration of such a gammadelta T-cell(s) or gammadelta T-cell population can also be treated with another cancer therapy, such as chemotherapy, radiation, or with a combination of both, concomitantly with receiving a gammadelta T-cell or gammadelta T-cell population of the disclosure. In other embodiments, administration of such a gammadelta T-cell(s) or gammadelta T-cell population can be provided prior to another cancer therapy, such as chemotherapy, radiation, surgery or a combination thereof.

A population of expanded gammadelta T-cells can comprise two or more cells that express identical, different, or a combination of identical and different tumor recognition moieties.

In one embodiment, the expanded gammadelta T-cell enriched cell population is administered in an amount effective to reduce or eliminate a cancer, such as a solid tumor, metastatic cancer or a malignancy, or prevent its occurrence or recurrence. “An amount effective to reduce or eliminate the solid tumor or to prevent its occurrence or recurrence” or “an amount effective to reduce or eliminate the hyperproliferative disorder or to prevent its occurrence or recurrence” refers to an amount of a therapeutic composition that improves a patient outcome or survival following treatment for the tumor disease state or hyperproliferative disorder as measured by patient test data, survival data, elevation or suppression of tumor marker levels, reduced susceptibility based upon genetic profile or exposure to environmental factors. “Inhibiting tumor growth” refers to reducing the size or viability or number of cells of a tumor. “Cancer”, “malignancy”, “solid tumor” or “hyperproliferative disorder” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features. A “cancerous” or “malignant cell” or “solid tumor cell” is understood as a cell having specific structural properties, lacking differentiation and being capable of invasion and metastasis. “Cancer” refers to all types of cancer or neoplasm or malignant tumors found in mammals, including carcinomas and sarcomas. Examples are cancers of the breast, lung, non-small cell lung, stomach, brain, head and neck, medulloblastoma, bone, liver, colon, genitourinary, bladder, urinary, kidney, testes, uterus, ovary, cervix, prostate, melanoma, mesothelioma, sarcoma, (see DeVita, et al., (eds.), 2001, Cancer Principles and Practice of Oncology, 6th. Ed., Lippincott Williams & Wilkins, Philadelphia, Pa.; this reference is herein incorporated by reference in its entirety for all purposes).

“Cancer-associated” refers to the relationship of a nucleic acid and its expression, or lack thereof, or a protein and its level or activity, or lack thereof, to the onset of malignancy in a subject cell. For example, cancer can be associated with expression of a particular gene that is not expressed, or is expressed at a lower level, in a normal healthy cell. Conversely, a cancer-associated gene can be one that is not expressed in a malignant cell (or in a cell undergoing transformation), or is expressed at a lower level in the malignant cell than it is expressed in a normal healthy cell.

“Hyperproliferative disease” refers to any disease or disorder in which the cells proliferate more rapidly than normal tissue growth. Thus, a hyperproliferating cell is a cell that is proliferating more rapidly than normal cells.

“Advanced cancer” means cancer that is no longer localized to the primary tumor site, or a cancer that is Stage III or IV according to the American Joint Committee on Cancer (AJCC).

“Well tolerated” refers to the absence of adverse changes in health status that occur as a result of the treatment and would affect treatment decisions.

“Metastatic” refers to tumor cells, e.g., human solid tumor or genitourinary malignancy, that are able to establish secondary tumor lesions in the lungs, liver, bone or brain of immune deficient mice upon injection into the mammary fat pad and/or the circulation of the immune deficient mouse.

A “solid tumor” includes, but is not limited to, sarcoma, melanoma, carcinoma, or other solid tumor cancer. “Sarcoma” refers to a tumor which is made up of a substance like the embryonic connective tissue and is generally composed of closely packed cells embedded in a fibrillar or homogeneous substance. Sarcomas include, but are not limited to, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

“Melanoma” refers to a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungual melanoma, and superficial spreading melanoma.

“Carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidernoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma viflosum.

“Leukemia” refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease—acute or chronic; (2) the type of cell involved; myeloid (myelogenous), lymphoid (lymphogenous), or monocytic; and (3) the increase or non-increase in the number of abnormal cells in the blood—leukemic or aleukemic (subleukemic). Leukemia includes, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia. Additional cancers that may be treated or prevented with the methods, compositions or gammadelta T-cell enriched cell populations of the present invention include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

In another particular embodiment of this aspect of the present invention the method is affected concomitantly with, following or prior to hematopoietic, hematopoietic progenitor or hematopoietic stem cell transplantation into said subject.

In specific embodiments, the expanded gammadelta T-cells of the invention are transplanted (e.g. infused) into a subject prior to, concomitantly with or following treatment with ex-vivo expanded hematopoietic stem cells.

In some particular embodiments, the subject in need thereof is a past, present or future recipient of hematopoietic stem cells expanded by culturing with greater than 1.0 mM nicotinamide. Protocols for preparation and treatment of patients with such a population of hematopoietic stem cells expanded with millimolar concentrations of nicotinamide (e.g. NiCord™, Gamida-Cell, Jerusalem, Israel) are described in detail in International Patent Application Nos: WO2018211487 and WO2018211509, and U.S. Pat. Nos. 7,955,852, 8,187,876 and 8,846,393. In specific embodiments, the subject in need thereof is a patient following treatment with NiCord™. In yet another specific embodiment, the subject in need thereof is a patient about to be treated with NiCord™, or currently being treated with NiCord™ In still another embodiment, the subject in need thereof is in remission from a cancer following treatment with NiCord™.

In yet further embodiments, the subject is being concomitantly treated with a sensitizing or potentiating agent (e.g., proteasome inhibitor, IL-2, IL-15, etc) further enhancing the in-vivo function of the transfused gammadelta T-cell enriched cells.

Decreased numbers and functionality of gammadelta T-cells in autoimmune patients has been observed, indicating the possibility of gammadelta T-cell therapy in a variety of autoimmune diseases and conditions. Thus, in still another embodiment of the present invention there is provided a method of treating an autoimmune disease or condition in a subject in need thereof. The method according to this aspect of the present invention is effected by administering a therapeutic amount of a population of gammadelta T-cells of the invention to said subject.

Autoimmune diseases which can be treated by the method and/or compositions of the invention include, but are not limited to cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis, myocardial infarction, thrombosis, Wegener's granulomatosis, Takayasu's arteritis, Kawasaki syndrome, anti-factor VIII autoimmune disease, necrotizing small vessel vasculitis, microscopic polyangiitis, Churg and Strauss syndrome, pauci-immune focal necrotizing and crescentic glomerulonephritis, antiphospholipid syndrome, antibody-induced heart failure, thrombocytopenic purpura, autoimmune hemolytic anemia, cardiac autoimmunity in Chagas' disease and anti-helper T lymphocyte autoimmunity.

Examples of autoimmune rheumatoid diseases include, but are not limited to rheumatoid arthritis and ankylosing spondylitis.

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. diseases include, but are not limited to autoimmune diseases of the pancreas, Type 1 diabetes, autoimmune thyroid diseases, Graves' disease, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome.

Examples of autoimmune gastrointestinal diseases include, but are not limited to, chronic inflammatory intestinal diseases, celiac disease, colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis, primary biliary cirrhosis and autoimmune hepatitis.

Examples of autoimmune neurological diseases include, but are not limited to, multiple sclerosis, Alzheimer's disease, myasthenia gravis, neuropathies, motor neuropathies; Guillain-Barre syndrome and autoimmune neuropathies, myasthenia, Lambert-Eaton myasthenic syndrome; paraneoplastic neurological diseases, cerebellar atrophy, paraneoplastic cerebellar atrophy and stiff-man syndrome; non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies; dysimmune neuropathies; acquired neuromyotonia, arthrogryposis multiplex congenita, neuritis, optic neuritis and neurodegenerative diseases.

Examples of autoimmune muscular diseases include, but are not limited to, myositis, autoimmune myositis and primary Sjogren's syndrome and smooth muscle autoimmune disease.

Examples of autoimmune nephric diseases include, but are not limited to, nephritis and autoimmune interstitial nephritis.

Examples of autoimmune diseases related to reproduction include, but are not limited to, repeated fetal loss.

Examples of autoimmune connective tissue diseases include, but are not limited to, ear diseases, autoimmune ear diseases and autoimmune diseases of the inner ear.

Examples of autoimmune systemic diseases include, but are not limited to, systemic lupus erythematosus and systemic sclerosis.

In some cases, methods of the present invention expanded gammadelta T-cells or a gammadelta T-cell population of the disclosure may be used to treat an infectious disease. The method according to this aspect of the present invention is effected by administering a therapeutic amount of the cultured gammadelta T-cell enriched cells of the invention to a subject. An infectious disease may be caused, for example, by a pathogenic bacterium or by a virus. Various pathogenic proteins, nucleic acids, lipids, or fragments thereof can be expressed by a diseased cell. An antigen presenting cell can internalize such pathogenic molecules, for instance with phagocytosis or by receptor-mediated endocytosis, and display a fragment of the antigen bound to an appropriate MHC molecule. Expanded gammadelta T-cells of the disclosure may recognize various antigens and antigen fragments of a pathogenic bacterium or a virus. Non-limiting examples of pathogenic bacteria can be found in the: a) Bordetella genus, such as Bordetella pertussis species; b) Borrelia genus, such Borrelia burgdorferi species; c) Brucella genus, such as Brucella abortus, Brucella canis, Brucella melitensis, and/or Brucella suis species; d) Campylobacter genus, such as Campylobacter jejuni species; e) Chlamydia and Chlamydophila genuses, such as Chlamydia pneumonia, Chlamydia trachomatis, and/or Chlamydophila psittaci species; f) Clostridium genus, such as Clostridium botulinum, Clostridium difficile, Clostridium perfringens, Clostridium tetani species; g) Corynebacterium genus, such as Corynebacterium diphtheria species; h) Enterococcus genus, such as Enterococcus faecalis, and/or Enterococcus faecium species; i) Escherichia genus, such as Escherichia coli species; j) Francisella genus, such as Francisella tularensis species; k) Haemophilus genus, such as Haemophilus influenza species; l) Helicobacter genus, such as Helicobacter pylori species; m) Legionella genus, such as Legionella pneumophila species; n) Leptospira genus, such as Leptospira interrogans species; o) Listeria genus, such as Listeria monocytogenes species; p) Mycobacterium genus, such as Mycobacterium leprae, Mycobacterium tuberculosis, and/or Mycobacterium ulcerans species; q) Mycoplasma genus, such as Mycoplasma pneumonia species; r) Neisseria genus, such as Neisseria gonorrhoeae and/or Neisseria meningitidia species; s) Pseudomonas genus, such as Pseudomonas aeruginosa species; t) Rickettsia genus, such as Rickettsia rickettsii species; u) Salmonella genus, such as Salmonella typhi and/or Salmonella typhimurium species; v) Shigella genus, such as Shigella sonnei species; w) Staphylococcus genus, such as Staphylococcus aureus, Staphylococcus epidermidis, and/or Staphylococcus saprophyticus species; x) Streptococcus genus, such as Streptococcus agalactiae, Streptococcus pneumonia, and/or Streptococcus pyogenes species; y) Treponema genus, such as Treponema pallidum species; z) Vibrio genus, such as Vibrio cholera; and/or aa) Yersinia genus, such as Yersinia pestis species.

In some cases, methods and/or compositions of the present invention or the expanded gammadelta T-cell or gammadelta T-cell population of the disclosure may be used to treat an infectious disease, an infectious disease may be caused a virus. Non-limiting examples of viruses can be found in the following families of viruses and are illustrated with exemplary species: a) Adenoviridae family, such as Adenovirus species; b) Herpesviridae family, such as Herpes simplex type 1, Herpes simplex type 2, Varicella-zoster virus, Epstein-barr virus, Human cytomegalovirus, Human herpesvirus type 8 species; c) Papillomaviridae family, such as Human papillomavirus species; d) Polyomaviridae family, such as BK virus, JC virus species; e) Poxviridae family, such as Smallpox species; f) Hepadnaviridae family, such as Hepatitis B virus species; g) Parvoviridae family, such as Human bocavirus, Parvovirus B19 species; h) Astroviridae family, such as Human astrovirus species; i) Caliciviridae family, such as Norwalk virus species; j) Flaviviridae family, such as Hepatitis C virus (HCV), yellow fever virus, dengue virus, West Nile virus species; k) Togaviridae family, such as Rubella virus species; 1) Hepeviridae family, such as Hepatitis E virus species; m) Retroviridae family, such as Human immunodeficiency virus (HIV) species; n) Orthomyxoviridaw family, such as Influenza virus species; o) Arenaviridae family, such as Guanarito virus, Junin virus, Lassa virus, Machupo virus, and/or Sabia virus species; p) Bunyaviridae family, such as Crimean-Congo hemorrhagic fever virus species; q) Filoviridae family, such as Ebola virus and/or Marburg virus species; Paramyxoviridae family, such as Measles virus, Mumps virus, Parainfluenza virus, Respiratory syncytial virus, Human metapneumovirus, Hendra virus and/or Nipah virus species; r) Rhabdoviridae genus, such as Rabies virus species; s) Reoviridae family, such as Rotavirus, Orbivirus, Coltivirus and/or Banna virus species. In some examples, a virus is unassigned to a viral family, such as Hepatitis D.

Transplantation of hematopoietic cells has become the treatment of choice for a variety of inherited or malignant diseases. However, hematopoietic cell compositions are often rich in T lymphocytes, which contribute to graft-versus-host disease. Since patients suffering from hematological malignancies are often deficient in gammadelta T-cell numbers and function, exogenous administration gammadelta T-cells along with hematopoietic cell transplantation is currently being investigated for enhanced long term engraftment and prevention of graft versus host disease. Thus, in yet another embodiment of the present invention there is provided a method of treating or preventing graft versus host disease in a subject in need thereof. Thus, in still another embodiment of the present invention there is provided a method of treating an autoimmune disease or condition in a subject in need thereof. The method according to this aspect of the present invention is effected by administering a therapeutic amount of a population of gammadelta T-cells or compositions comprising same of the invention to said subject.

### Treatment Regimes

According to some aspects of some embodiments of the present invention, there are provided pharmaceutical compositions comprising a gammadelta T-cell enriched cell population of the invention for the treatment of disease, e.g., metastic cancer, solid tumors, autoimmune disease, hyperproliferative disorder or a viral infection, formulated together with a pharmaceutically acceptable carrier. Some compositions include a combination of multiple (e.g., two or more) gammadelta T-cell enriched cell populations of the invention.

In prophylactic applications, pharmaceutical compositions or medicaments are administered to a patient susceptible to, or otherwise at risk of a disease or condition (i.e., a hyperproliferative disease or solid tumor) in an amount sufficient to eliminate or reduce the risk of recurrence of the hyperproliferative disease or solid tumor, lessen the severity, or delay the outset of the disease, including biochemical, histologic and/or behavioral symptoms of the disease, its complications and intermediate pathological phenotypes presenting during development of the disease. In therapeutic applications, compositions or medicants are administered to a patient suspected of, or already suffering from such a disease in an amount sufficient to cure, or at least partially arrest, the symptoms of the disease (biochemical, histologic and/or behavioral), including its complications and intermediate pathological phenotypes in development of the disease. An amount adequate to accomplish therapeutic or prophylactic treatment is defined as a therapeutically- or prophylactically-effective dose. In both prophylactic and therapeutic regimes, agents are usually administered in several dosages until a sufficient anti-proliferative response has been achieved. Typically, the anti-proliferative response is monitored and repeated dosages are given if the anti-proliferative response starts to wane.

An expanded gammadelta T-cell(s) or gammadelta T-cell population as described herein can be administered before, during, or after the occurrence of a disease or condition (e.g. the onset of a cancer, an infectious disease, an immune disease, sepsis, or with a bone marrow transplant) and for a length of time necessary for the treatment of the, and the timing of administering a pharmaceutical composition containing an expanded gammadelta T-cell or gammadelta T-cell population can vary. For example, the expanded gammadelta T-cell or gammadelta T-cell population can be used as a prophylactic, can be administered to a subject during or as soon as possible after the onset of the symptoms or within any period of time from the onset of symptoms. For the treatment of cancer, for example, one or multiple dosages of the expanded gammadelta T-cell or gammadelta T-cell population can be administered years after onset of the cancer and before or after other treatments. The length of treatment can vary for each subject.

### Effective Dosages

Effective doses of a composition of a gammadelta T-cell population for the treatment of disease, e.g., metastic cancer, solid tumors, or a hyperproliferative disorder, described herein vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. Treatment dosages need to be titrated to optimize safety and efficacy.

For administration with a therapeutic gammadelta T-cell or gammadelta T-cell enriched cell population, the dosage ranges from about 1×10⁶ to about 1×10⁹ gammadelta T-cells and/or gammadelta T-cell enriched cells per patient. For administration with an gammadelta T-cell enriched cell population, the dosage ranges from about 1×10⁵ to about 1×10⁹ gammadelta T-cells and/or gammadelta T-cell enriched cells per kilogram recipient weight, or the dosage ranges from about 5×10⁵ to about 1×10⁸ gammadelta T-cells and/or gammadelta T-cell enriched cells per kilogram recipient weight. An exemplary treatment regime entails administration once per every two weeks or once a month or once every 3 to 6 months. In some methods, two or more gammadelta T-cell enriched cell populations are administered simultaneously, in which case the dosage of each gammadelta T-cell enriched cell populations administered falls within the ranges indicated. Multiple administrations of gammadelta T-cell enriched cell populations can occur. Intervals between single dosages can be weekly, monthly or yearly. Intervals can also be irregular as indicated by measuring blood levels of the gammadelta T-cell enriched cell population in the patient. Alternatively, the gammadelta T-cell enriched cell populations can be administered as a sustained release formulation, in which case less frequent administration is required. Dosage and frequency vary depending on the half-life of the gammadelta T-cell enriched cell populations in the patient. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

### Routes of Administration

Compositions of a therapeutic gammadelta T-cell enriched cell population for the treatment of disease, e.g., metastic cancer, solid tumors, or a hyperproliferative disorder, can be administered by intravenous, intravesicular, intrathecal, parenteral, topical, subcutaneous, oral, intranasal, intraarterial, intracranial, intraperitoneal, or intramuscular means. As a prophylactic/adjuvant or for treatment of disease, therapeutic gammadelta T-cell enriched cell populations target a hyperproliferative disorder or solid tumor, e.g., a genitourinary malignancy, and/or therapeutic treatment. The most typical route of administration of an immunogenic agent is subcutaneous although other routes can be equally effective. The next most common route is intramuscular injection. This type of injection is most typically performed in the arm or leg muscles. In some methods, agents are injected directly into a particular tissue where deposits have accumulated, for example intracranial injection. Intramuscular injection on intravenous infusion are preferred for administration of a gammadelta T-cell enriched cell population. In some methods, a particular therapeutic cell population is injected directly into the bladder.

### Formulation

Compositions of a gammadelta T-cell enriched cell population for the treatment of disease, e.g., metastic cancer, solid tumors, viral or other infection, inflammatory or a hyperproliferative disorder.

Compositions of a therapeutic gammadelta T-cell enriched cell population for the treatment of disease, e.g., metastic cancer, solid tumors, or a hyperproliferative disorder, are often administered as pharmaceutical compositions comprising an active therapeutic agent, i.e., and a variety of other pharmaceutically acceptable components. See, e.g., Alfonso R Gennaro (ed), Remington: The Science and Practice of Pharmacy, (Formerly Remington's Pharmaceutical Sciences) 20th ed., Lippincott, Williams & Wilkins, 2003, incorporated herein by reference in its entirety. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. An example of such diluent is X-vivo 20 media (Cambrex Bio Science, Walkersville, Md.) containing 10% heat inactivated human AB serum or 10% autologous serum. Further examples of such diluents are distilled water, physiological phosphate-buffered saline, Ringer's solutions, dextrose solution, and Hank's solution. In addition, the pharmaceutical composition or formulation can also include other carriers, adjuvants, or nontoxic, nontherapeutic, nonimmunogenic stabilizers and the like.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

For parenteral administration, compositions of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Therapeutic gammadelta T-cell enriched cell populations can be administered in the form of a depot injection or implant preparation which can be formulated in such a manner as to permit a sustained release of the active ingredient. An exemplary composition comprises a therapeutic gammadelta T-cell enriched cell population at 5 mg/mL, formulated in aqueous buffer consisting of 50 mM L-histidine, 150 mM NaCl, adjusted to pH 6.0 with HCl.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science, 249: 1527, 1990; Hanes, Advanced Drug Delivery Reviews, 28: 97-119, 1997, incorporated herein by reference in their entirety. The agents of this invention can be administered in the form of a depot injection or implant preparation which can be formulated in such a manner as to permit a sustained or pulsatile release of the active ingredient. Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

The pharmaceutical compositions generally comprise a composition of the therapeutic gammadelta T-cell enriched cell population in a form suitable for administration to a patient. The pharmaceutical compositions are generally formulated as sterile, substantially isotonic and in fall compliance with all Good Manufacturing Practice (GMP) regulations of the U.S. Food and Drug Administration.

### Toxicity

Preferably, a therapeutically effective dose of a composition of the gammadelta T-cell enriched cell population described herein will provide therapeutic benefit without causing substantial toxicity.

Toxicity of the therapeutic gammadelta T-cell enriched cell population described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the therapeutic gammadelta T-cell enriched cell population described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. (See, e.g., Fingl, et al., The Pharmacological Basis Of Therapeutics, Ch. 1, 1975), incorporated herein by reference in its entirety.

### Unit Dosage

An expanded gammadelta T-cell(s) or gammadelta T-cell population disclosed herein may be formulated in unit dosage forms suitable for single administration of precise dosages. In some cases, the unit dosage forms comprise additional lymphocytes, for example, but not limited to NK, or hematopoietic stem cells. In unit dosage form, the formulation is divided into unit doses containing appropriate quantities of one or more compounds. The unit dosage can be in the form of a package containing discrete quantities of the formulation. Non-limiting examples are packaged tablets or capsules, and powders in vials or ampoules. Aqueous suspension compositions can be packaged in single-dose non-reclosable containers. Multiple-dose reclosable containers can be used, for example, in combination with a preservative or without a preservative. In some examples, the cells, compositions or pharmaceutical composition do not comprise a preservative. Formulations for parenteral injection can be presented in unit dosage form, for example, in ampoules, or in multi-dose containers with a preservative.

### Kits

Also within the scope of the invention are kits comprising the compositions (e.g., a therapeutic gammadelta T-cell enriched cell population) of the invention and instructions for use. The kit can further contain a least one additional reagent, or one or more additional human antibodies of the invention (e.g., a human antibody having a complementary activity which binds to an epitope in the antigen distinct from the first human antibody). Kits typically include a label indicating the intended use of the contents of the kit. The term label includes any writing, or recorded material supplied on or with the kit, or which otherwise accompanies the kit.

### Cryopreservation

In some embodiments, gammadelta T-cells or gammadelta T-cell population(s) may be formulated in cryopreservation media and placed in cryogenic storage units such as liquid nitrogen freezers (−195 C) or ultra-low temperature freezers (−65 C, −80 C or −120 C) for long-term storage of at least about 1 month, 2 months, 3 months, 4 months, 5 months, 6 months, 1 year, 2 years, 3 years, or at least 5 years. The cryopreservation media can contain dimethyl sulfoxide (DMSO), and/or sodium chloride (NaCl), and/or dextrose, and/or dextran sulfate and/or hydroyethyl starch (HES) with physiological pH buffering agents to maintain pH between about 6.0 to about 6.5, about 6.5 to about 7.0, about 7.0 to about 7.5, about 7.5 to about 8.0 or about 6.5 to about 7.5. The cryopreserved gammadelta T-cells or gammadelta T-cell population(s) can be thawed and further processed by stimulation with antibodies, proteins, peptides, and/or cytokines as described herein.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6.

This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### EXPERIMENTAL PROCEDURES

Ex-Vivo Culture of Gammadelta T-Cells

Healthy donor blood units samples were depleted of TCR alphabeta-expressing T-cells using midiMACS™ columns and TCRα/β Kit (Miltenyi Biotec, Gaithersberg, Md.). Alphabeta-T-cell depleted populations were cultured for 12-13 days in VueLife® (Saint-Gobain, Gaithersburg, Md.) bags with medium supplemented with 0 (control) or 5 mM nicotinamide (NAM) and 50 ng/ml IL-2.

T-Cell Receptor Characterization

To characterize T cell receptor (TCR) expressed on CD3+ cells in culture, CD3 positive cells were purified with CliniMACS™ CD3 reagent (Miltenyi, 273-01, Gaithersburg, Md.) followed by FACS analysis of the percentages of CD3+/γδ+ and CD3+/α/β+ cells. FACS staining and analysis was performed according to standard procedures employing cell surface-specific antibodies.

CD62L Expression in Expanded GammaDelta T-Cells

Purified gamma-delta T-cells from NAM-treated and control (−NAM) cultures were stained for CD62L (L-selectin) and analyzed by FACS, using anti-CD62L antibodies.

Transplantation and In-Vivo Functionality of Expanded GammaDelta T-Cells

CD3+/gammadelta+ cells were purified from culture, as described above, and then marked with CFSE (Invitrogen, Thermo-Fisher, Carlsbad, Calif.). NSG SCID mice were irradiated at 350cGy. The next day following irradiation, 5-6×10⁶ CFSE-labeled CD3+/gammdelta-positive cells per mouse from NAM and control (−NAM) cultures were injected intravenously into the mice.

Mice were sacrificed 4 days after cell infusion, and organs excised. Fractions of CFSE stained cells were evaluated by FACS of organ cell suspensions.

### Example 1: Ex-Vivo Culture of Human Gamma-Delta T-Cells with NAM Enhances T-Cell Functionality

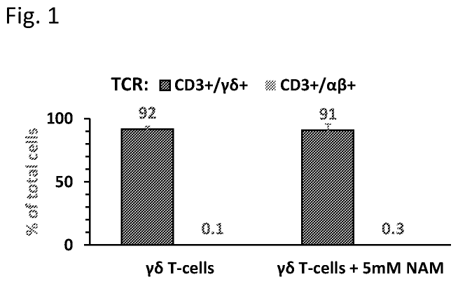

FACS analysis of alphabeta-depleted T-cells cultured with 5 mM nicotinamide (NAM) showed that depletion of the alphabeta T-cell fraction provides an expanded CD3+ cell population comprising greater than 90% gammadelta T-cells. Culturing the cells with nicotinamide does not seem to affect the expansion of the gammadelta positive component of the cultured T-cell populations (FIG. 1).

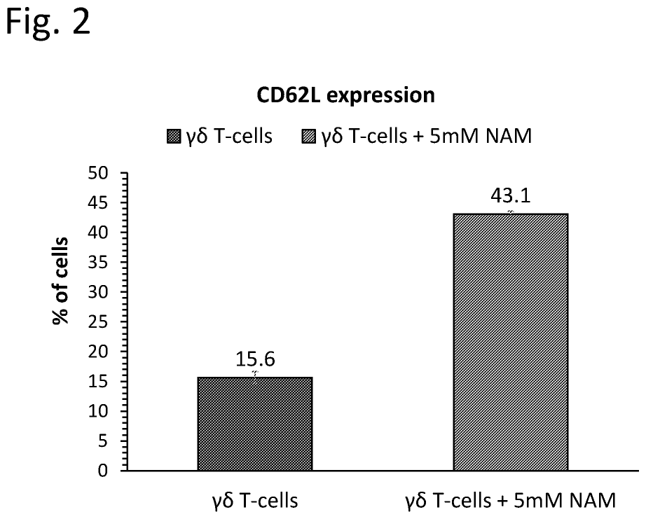

CD62L (L-selectin) is an important lymphocyte adhesion molecule, acting as a “homing receptor” for homing and entrance of lymphocytes into lymphoid tissue as well as a T-lymphocyte co-stimulatory signal. FIG. 2 shows the striking enhancement of CD62L expression (detected by FACS) in gammadelta T-cells cultured with 5 mM NAM, compared with identical cells cultured without NAM.

### Example 2: Enhanced In-Vivo Homing and Retention of Gammadelta T-Cells Cultured with NAM

Low frequencies of effective tissue homing and retention of infused lymphocytes constitutes a serious obstacle to successful T-cell therapies, increasing the numbers of cells required to achieve optimal results. Increased functionality of NAM-cultured gammadelta T-cells is reflected in enhanced incidence of homing and retention in tissues after infusion into irradiated scid NSG mice.

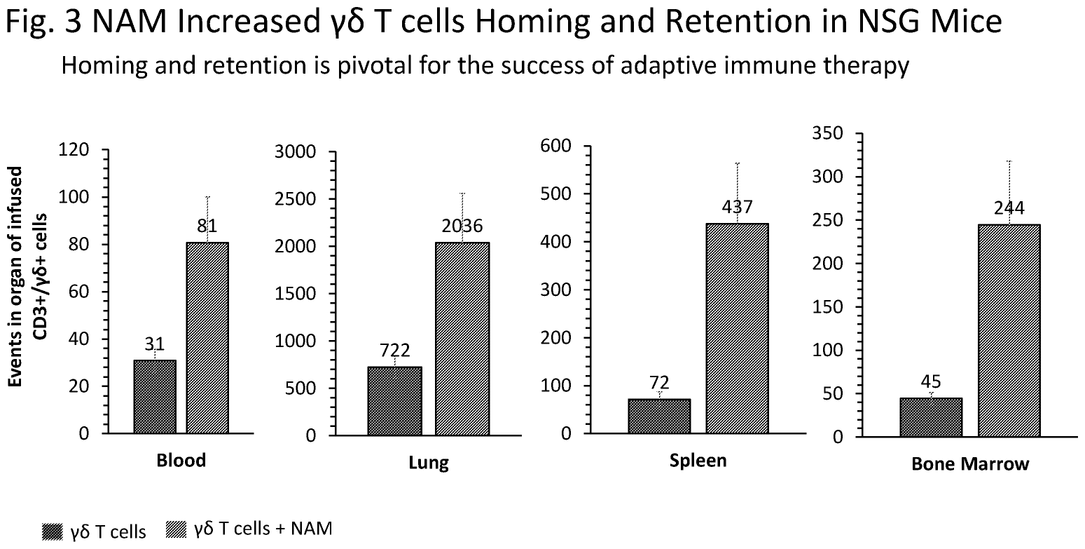

FIG. 3 shows the magnitude of NAM's effect on in-vivo homing and retention 4 days after infusion of the NAM-cultured gammadelta T-cells. Of particular significance is the powerful enhancement of gammadelta T-cells retained in lymphoid tissue (spleen, bone marrow), alongside the nearly three-fold increase in gammadelta T-cells retained in blood and lung tissue resulting from culture with NAM.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1. A method of enhancing gammadelta T-cell homing and/or retention potential, the method comprising:
(a) obtaining a selected cell population enriched for gammadelta T-cells;
(b) ex-vivo providing said selected cell population with conditions for gammadelta T-cell expansion, comprising providing nutrients and cytokines, wherein said cytokines are selected from the group consisting of IL-2, IL-15 and IL-21;
(c) providing nicotinamide in the range of 0.5 to 50 mM for a period of time sufficient for enhancing gammadelta T-cell homing and/or retention potential,
thereby enhancing homing and/or retention potential of gammadelta T-cells in said selected cell population.

2. A method of enhancing gammadelta T-cell CD62L expression, the method comprising:
(a) obtaining a selected cell population enriched for gammadelta T-cells;
(b) ex-vivo providing said selected cell population with conditions for gammadelta T-cell expansion, comprising providing nutrients and cytokines, and wherein said cytokines are selected from the group consisting of IL-2, IL-15 and IL-21;
(c) providing nicotinamide in the range of 0.5 to 50 mM for a period of time sufficient for enhancing gammadelta T-cell CD62L expression,
thereby enhancing CD62L expression of gammadelta T-cells in said selected cell population.

3.-5. (canceled)

6. The method of claim 1, wherein said selected cell population is a lymphocyte cell population enriched for gammadelta T-cells by alphabeta T-cell depletion and/or selection of gammadelta T-cells.

7. The method of claim 6, wherein said selected cell population is a lymphocyte cell populations enriched for gammelta T-cells by alphabeta T-cell depletion and said selected cell population comprises natural killer (NK) cells, and wherein method further comprises providing conditions for NK cell expansion.

8.-11. (canceled)

12. The method of claim 1, wherein said population of gammadelta T-cells is derived from an organ selected from the group consisting of a muscle, skin, a bone, a lymph organ, a pancreas, a liver, a gallbladder, a kidney, a digestive tract organ, a respiratory tract organ, a reproductive organ, a urinary tract organ, a blood-associated organ, a thymus, a spleen, a nervous system organ.

13. The method of claim 1, wherein said population of gammadelta T-cells is derived from a source selected from the group consisting of hematopoietic cells, umbilical cord blood cells, mobilized peripheral blood cells and bone marrow cells.

14.-15. (canceled)

16. The method of claim 1, wherein said population of cells is derived from a mononuclear cell fraction or an apheresis sample.

17. (canceled)

18. The method of claim 1, wherein said period of time of step (c) is between 1 and 3 weeks, or between 1 and 7 days.

19.-20. (canceled)

21. The method of claim 1, wherein said nicotinamide is provided at a concentration of 5 mM or 7 mM.

22. The method of claim 1, further comprising selecting a gammadelta T-cell population according to a cell marker selected from the group consisting of a tumor antigen, a viral antigen and a bacterial antigen.

23. A therapeutic cell composition comprising an expanded selected gammadelta T-cell population, said expanded cell population ex-vivo cultured with conditions for gammadelta T-cell expansion and amount of nicotinamide in the range of 0.5-50 mM, wherein said expanded selected gammadelta T-cell population is characterized by at least one of:
(i) enhanced gammadelta T-cell homing and/or retention potential, and
(ii) enhanced expression of CD62L,
as compared to a similar selected gammadelta T-cell population expanded with identical conditions and no more than 0.1 mM nicotinamide.

24. The therapeutic cell composition of claim 23, comprising gammadelta T-cells cultured according to the method comprising:
(a) obtaining a selected cell population enriched for gammadelta T-cells;
(b) ex-vivo providing said selected cell population with conditions for gammadelta T-cell expansion,
(c) providing nicotinamide in the range of 0.5 to 50 mM for a period of time sufficient for enhancing gammadelta T-cell homing and/or retention potential.

25. The therapeutic cell composition of claim 23, further comprising NK cells.

26. A method of transplanting cells in a human subject, the method comprising:
(a) ex-vivo expanding a selected gammadelta T-cell population by culturing said cell population conditions for gammadelta T-cell expansion and nicotinamide in the range of 0.5-50 mM for a period of time sufficient for enhancing gammadelta T-cell homing and/or retention potential and/or CD62L expression, wherein said expanded selected gammadelta T-cell population is characterized by at least one of:
(i) enhanced gammadelta T-cell homing and/or retention potential, and
(ii) enhanced CD62L expression,
as compared to a similar selected gammadelta T-cell population expanded without 0.5-50 nM nicotinamide, and
(b) infusing the expanded gammadelta T-cells into a subject in need thereof.

27. The method of claim 26, wherein step (a) is affected according to the method comprising:
(i) obtaining a selected cell population enriched for gammadelta T-cells;
(ii) ex-vivo providing said selected cell population with conditions for gammadelta T-cell expansion,
(iii) providing nicotinamide in the range of 0.5 to 50 mM for a period of time sufficient for enhancing gammadelta T-cell homing and/or retention potential.

28. (canceled)

29. The method of claim 26, wherein said gammadelta T-cells are allogeneic to said subject.

30. The method of claim 26, wherein said gammadelta T-cells are autologous to said subject.

31. The method of claim 26, wherein said subject is suffering from a condition selected from the group consisting of a cancer, a bacterial infection, a viral infection, an autoimmune condition and an inflammatory condition.

32. The method of claim 31, wherein transplantation of said cells in said subject comprises an adjunct therapy, and wherein said adjunct therapy is in combination with a therapy selected from the group consisting of anti-viral therapy, anti-inflammatory therapy, antibiotic therapy, bactericidal therapy, chemotherapy, surgery, immunotherapy, immunochemotherapy, radiotherapy, bone marrow transplantation and hematopoietic stem cell transplantation.

33. (canceled)

34. The method of claim 32, wherein said subject is being treated with umbilical cord blood hematopoietic stem cells expanded in culture with greater than 1.0 mM nicotinamide prior to, concomitantly with or following transplantation of said expanded gammadelta T-cells.
